(19) United States
(12) Patent Application Publication
(10) Pub. No.: US 2003/0191385 A1
(43) Pub. Date: Oct. 9, 2003

(54) Catheter guide assembly

(76) Inventors: Peter Hanley, Gloucestershire (GB); Ian Leitch Mcdougall, Oxon (GB)

Correspondence Address: STAAS & HALSEY LLP, SUITE 700, 1201 NEW YORK AVENUE, N.W., WASHINGTON, DC, 20005, US

(21) Appl. No.: 10/297,466

(22) PCT Filed: May 29, 2001
(86) PCT No.: PCT/GB01/02379

(30) Foreign Application Priority Data
Jun. 8, 2000 (GB) 0014028.5

(51) Int. Cl. A61B 5/05

(52) U.S. Cl. 600/410

(57) ABSTRACT

A catheter guide assembly (3) is provided having a magnet support for mounting to a catheter; at least two permanent magnets (1A+1B, 2) mounted to the support; and a control system (4). The control system (4) is used to adjust the relative magnetisation directions of the permanent magnets between a first configuration in which their direction of magnetisation are such that the magnets generate a relatively large resultant magnetic field, and a second configuration in which their directions of magnetisation are such that the magnets generate a relatively small resultant magnetic field.

The invention relates to a catheter guide assembly.

Catheterisation is a surgical procedure which is regarded as being minimally invasive and hence less traumatic than more open techniques. Applications include the repair of aneurisms and the removal of obstructions in blood vessels. For example, the treatment of coronary artery occlusions by Catheterisation is now a relatively common procedure in cardiology.

Traditionally, guidance is by the surgeon's manipulating the catheter, and the process is monitored using either ultrasound imaging or X-ray fluoroscopy. Ultrasound suffers from poor spatial resolution and the images are difficult to interpret. X-rays, because of the need for nearly continuous monitoring, involve large radiation doses both for the patient and also for the operator. For this reason, there is growing interest in “interventional MRI” where magnetic resonance imaging is used for monitoring the procedure.

Guidance of the catheter can be extremely difficult and time-consuming when the anatomy of the arterial system is complex, and this increases the trauma to the patient and the cost of the procedure.

Stereotaxis Inc has introduced a magnetically guided catheter system. The catheter has a magnetised tip, and this is directed by means of a system of superconducting coils which produce magnetic fields and field gradients appropriate to guiding the tip in the required direction. This system has been used in neurosurgery (see “Magnet on the Brain” Scientific American, August 1996). In this application, navigation is done with reference to a previously performed MRI scan, with markers included. There is periodic checking using X-ray fluoroscopy.

However, the use of magnetic guidance, while allowing navigation of complex anatomies, prevents the use of MRI for monitoring, because the magnetic field due to the catheter tip produces extreme distortion in the region of interest in an MRI scan.

In accordance with one aspect of the present invention, a catheter guide assembly comprises a magnet support for mounting to a catheter; at least two permanent magnets mounted to the support; and a control system for adjusting the relative magnetisation directions of the permanent magnets between a first configuration in which their directions of magnetisation are such that the magnets generate a relatively large resultant magnetic field, and a second configuration in which their directions of magnetisation are such that the magnets generate a relatively small resultant magnetic field.

In accordance with a second aspect of the present invention, a method of performing catheterisation comprises inserting a catheter assembly including a catheter and a catheter guide assembly according to the first aspect of the invention mounted to the catheter, with the magnetisations of the permanent magnets set to a first configuration; applying a magnetic field or magnetic field gradient in a controlled manner to cause the catheter to be guided by the catheter guide assembly; setting the magnetisations of the permanent magnets of the catheter guide assembly to their second configuration; removing the controlling magnetic field or magnetic field gradient; and performing a magnetic resonance imaging (MRI) process.

This invention enables the known advantages of permanent magnet catheter guide assemblies to be utilised, while at the same time the distorting magnetic field due to the permanent magnets can be “switched off” to allow an MRI process to be carried out. Typically, a separate magnet system is used for the MRI process.

It should also be noted that the presence of non-magnetic metal will not be a problem since radiologists allow MRI scans of patients with metal implants, prostheses etc.

In the preferred arrangement, the directions of magnetisation of the permanent magnets in the first configuration are substantially parallel, while the directions of magnetisation in the second configuration oppose one another so that the resultant magnetic field substantially zero it. However, it is envisaged that complete cancellation in the second configuration may not be necessary.

Typically, three permanent magnets will be provided, and in this case two may be fixed to the support while the other is moveable relative to the support, so as to control the two configurations. However, other numbers of magnets such as 2, 4, 5 or even more, could be used.

The magnets may be held relative to one another in a variety of ways. For example, they could be directly mounted to each other, but conveniently the permanent magnets are held by part of the support which passes around the permanent magnets so as to hold them in place, but permits the relative movement. For example, in the preferred approach, the magnets define a sphere and the support defines a corresponding part spherical enclosure in which the magnets are mounted.

The relative movement between the magnets will typically be controlled by at least one elongate control line, connected to one of the permanent magnets so that the permanent magnet can be moved by pulling and/or pushing the control line. In the preferred approach, a pair of control lines are provided for controlling movement in opposite directions.

In another approach, the magnetisation direction may be altered electronically. Thus, an electrical coil may be provided around one or more of the permanent magnets, the passage of an electric current through the coil causing the magnetisation of the magnet to change.

Some examples of catheter guide assemblies in accordance with the present invention will now be described with reference to the accompanying drawings, in which:—

FIG. 1 is a perspective view, partially cut away of a first example;

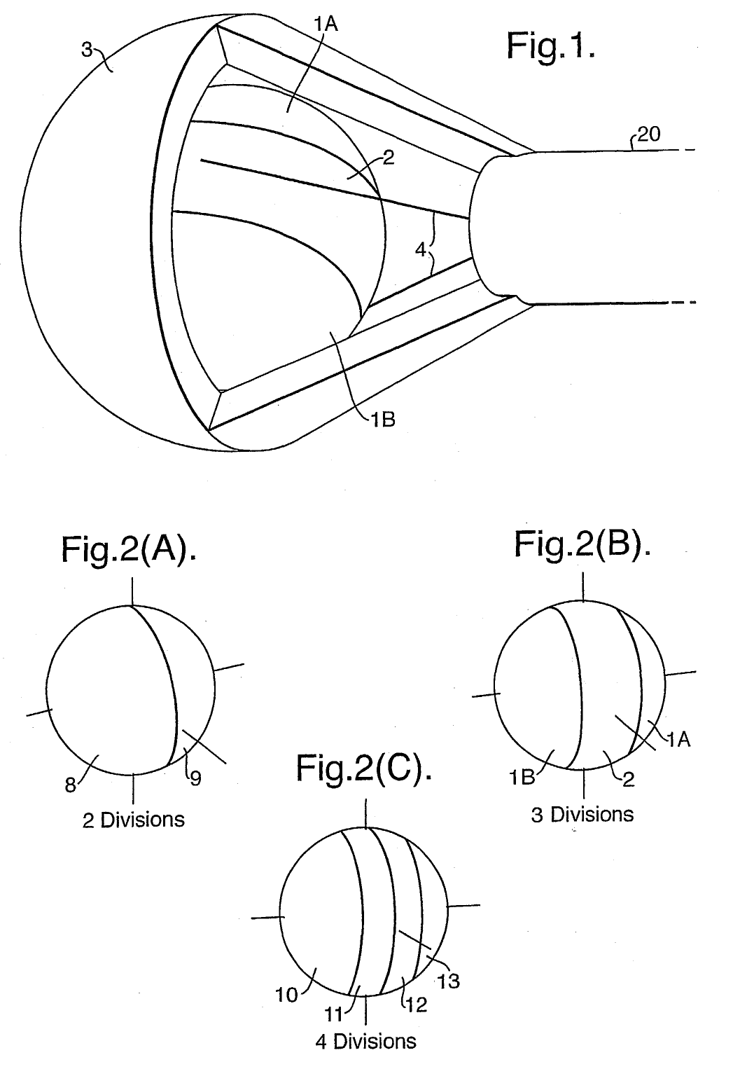

FIGS. 2A and 2C illustrate alternative permanent magnetic configurations, while FIG. 2B illustrates the permanent magnetic configuration of FIG. 1;

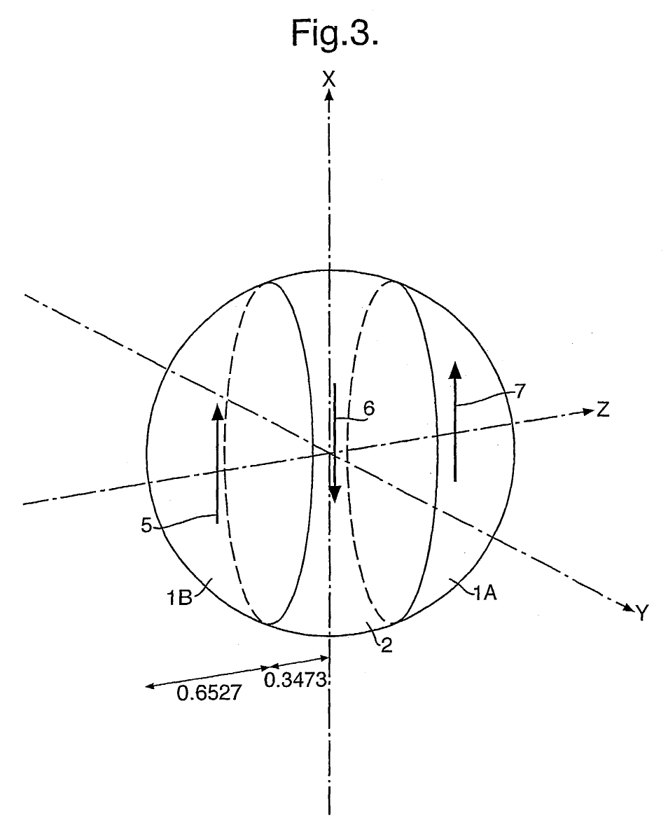

FIG. 3 illustrates schematically the permeant magnetic system of FIG. 1, showing the magnetisation directions and dimensions;

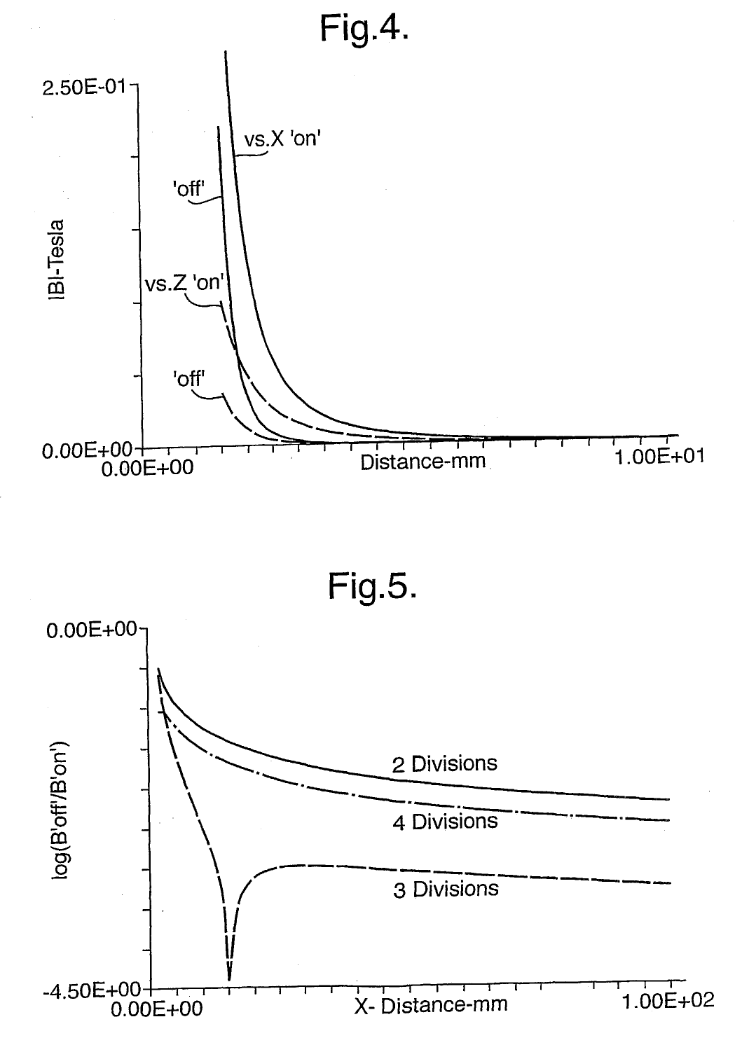

FIG. 4 illustrates graphically the variation in magnetic field strength in the X and Z directions of the FIG. 3 example in both its “on” and “off” configurations; and,

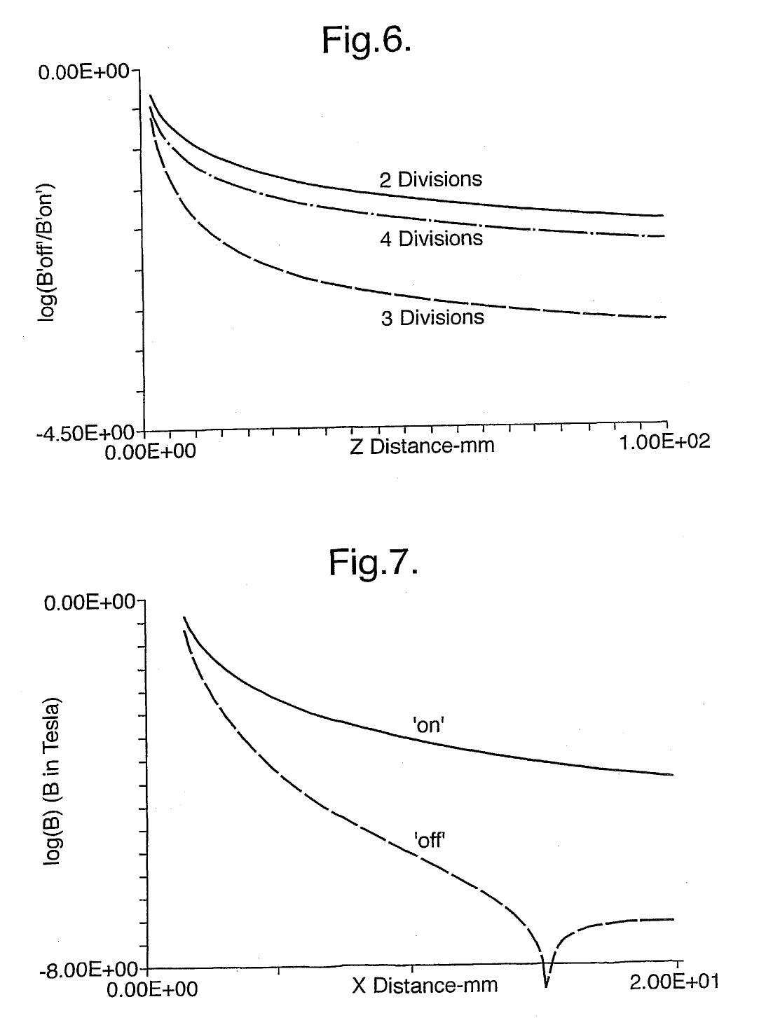

FIGS. 5 and 6 illustrate graphically the logarithm of the ratios of magnetic field strengths in the “off” and “on” configuration for magnet systems with 2, 3 and 4 permanent magnets in the X and Z directions respectively;

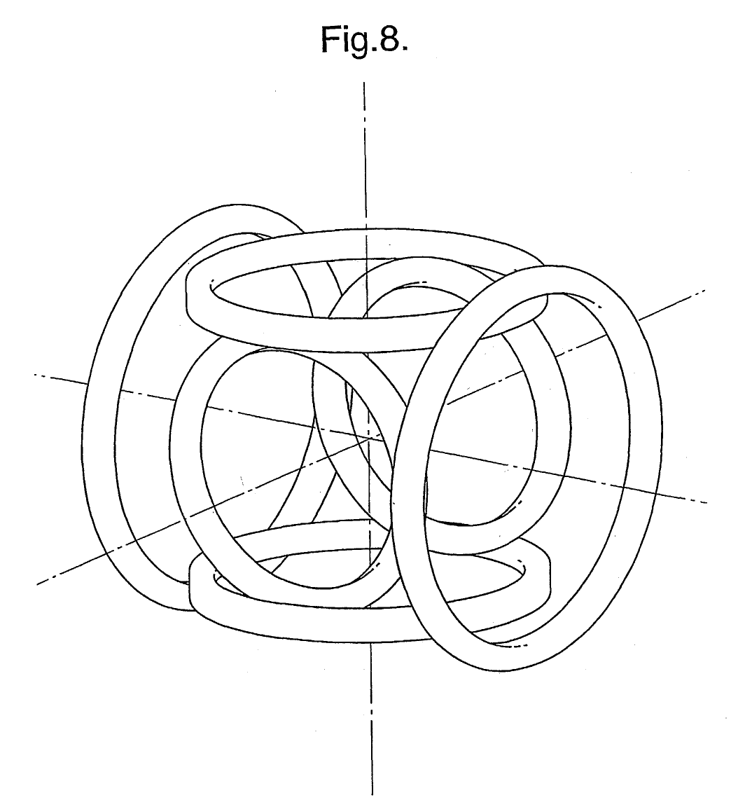

FIG. 7 shows the X data of FIG. 4 plotted logarithimically;

FIG. 8 illustrates a steering coil set; and,

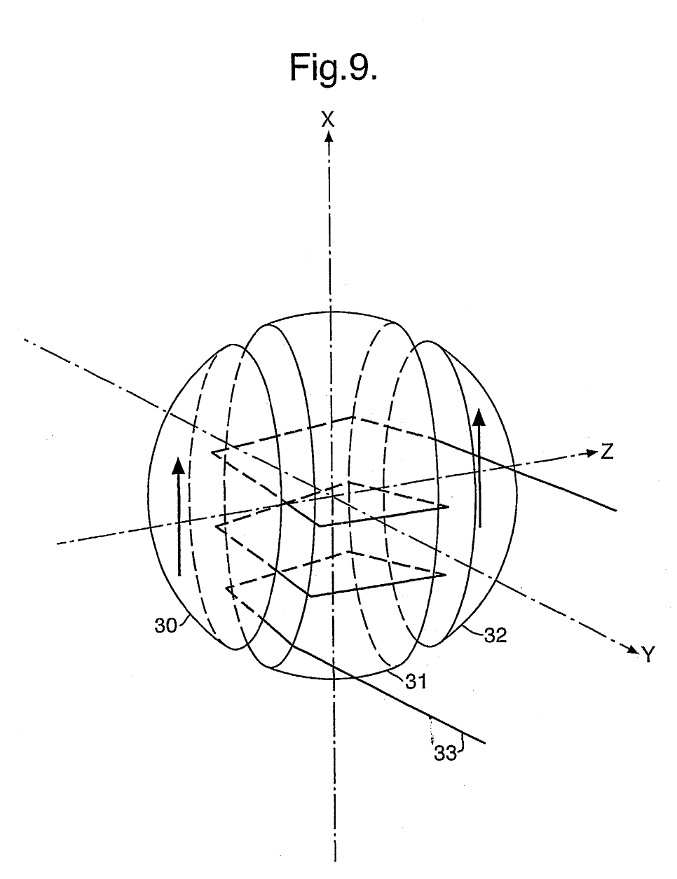

FIG. 9 illustrates a further example.

The assembly shown in FIG. 1 comprises a set of 3 permanent magnets 1A, 1B, 2 contacting each other and formed into a sphere, the assembly being supported in a part spherical housing 3. The central magnet 2 of the assembly can be rotated by means of a pair of cords 4 attached to the periphery of the permanent magnet 2, and extending back through a catheter 20 so that they can be operated by a surgeon.

Typically, each of the magnets 1A, 1B, 2 will be made of a magnetically hard material such as SmCo or NeFeB. FIG. 3 illustrates the assembly schematically and as can be seen this has a radius of 1 mm. The directions of magnetisation 5-7 are parallel. By rotating the permanent magnet 2, its direction of magnetisation 6 can be moved to be either opposite to the directions 5, 7 as shown in FIG. 3, or parallel to those directions. By suitably choosing the volumes of the magnets to be in inverse proportion to the strengths of their magnetisations, it can arranged that when the direction of magnetisation 6 is opposite to that of the magnetisations 5, 7 the resultant magnetic field will be significantly reduced, as will be explained in more detail below. It should be noted that because the material is magnetically hard the change and alignment has little effect on the strength of the magnetisation. The magnetisation is typically about 1.03 Webers/m².

Although 3 permanent magnets have been shown in FIGS. 1 and 3, other configurations are possible such as two hemispherical permanent magnets 8, 9 (FIG. 2A) or 4 permanent magnets 10-13 as shown in FIG. 2C.

It should be noted that in all the examples, the volumes of the opposing directions of magnetisation are equal. That is, if the volumes of the divisions or separate permanent magnets are chosen so that the total volume magnetised in one direction is equal to that magnetised in the opposition situation, the resultant magnetic field strength at some distance from the assembly is greatly reduced.

In order to demonstrate the effect of the invention in its configurations, a number of mathematical calculations have been performed. Field strengths were calculated by numerical integration over the magnetised volume (which were assumed to be uniformly magnetised) using the relationships:

\overline{B}=∇φ

$$φ = \frac{1}{4 π} \int \overline{M} \cdot {∇}_{q} \frac{1}{\vert \overline{q} - \overline{p} \vert} ⅆ V$$

where q is the source point and p is the field point. The fields were plotted against distance in the “X” and “Z” directions (as defined by FIG. 3).

The results for the “ON” and “OFF” conditions of the 3-division example are shown in FIG. 4.

As can be seen in FIG. 4, when the magnet configuration is as shown in FIG. 3 with direction of magnetisation opposed, the resultant magnetic field drops to zero in both the X and Z directions at about 3.5 mm from the centre of the magnet assembly. In contrast, when the magnetisations are aligned in the “on” configuration then there is a substantial resultant external magnetic field at this distance which can be used to steer the assembly and catheter. To get a more comprehensive view, FIGS. 5 and 6 show the logarithm of the ratios “OFF” to “ON” for 2, 3 and 4 division tips, plotted against “X” and “Z” respectively. These gibe an indication of the effectiveness of turning the field off. Thus a value of −2.0 on the ordinate means that the field strength has been reduced by a factor of 100.

It can be seen that the symmetry of “3 divisions” produces a greater reduction in field strength than does the uneven symmetry of “2” and “4 divisions”.

In order to be effective, when the magnetic tip is switched “OFF” for MRI, the field excursions should be small enough not to produce too much distortion in the image. Typically, in clinical MRI imaging gradients of the order of 10 mT/mm are desired.

FIG. 7 shows the “X” data of FIG. 4 plotted logarithmically. Prom this, it can be seen that a field of 10 mT is reached at a distance of about 7.5 mm from the tip. This implies that the distortion in the image would be less than 1 mm at this distance from the centre of the tip.

In use, a surgeon will insert a catheter 20 in a conventional manner into a human or animal body, and the body will be located within a conventional MRI magnetic system. The surgeon can then control gradient magnetic field coils such as shown in FIG. 8 also located within the whole body or open access MRI system, so as to draw the catheter through the body along an artery or the like, as a result of the interaction between the permanent magnets in the catheter guide assembly and the externally applied relevant field. During this process, the assembly will be in its first configuration with the magnetisations 5-7 aligned in the same direction. When the surgeon wishes to obtain an MRI image of the relevant part of the body where the catheter guide assembly is located, he operates the cords 4 so as to rotate the permanent magnet 2 about the Z axis to the (second) configuration shown in FIG. 3, so that the resultant magnetic field externally of the permanent magnets is substantially reduced as shown in FIG. 4. He can then arrange for the MRI process to be carried out.

As an additional option, a camera could be located on the end of the catheter 20 to enable he surgeon to view the insertion procedure.

FIG. 9 illustrates an alternative example comprising 3 permanent magnets 30-32 arranged in spherical form, the magnets 30-32 having their magnetisation directions parallel as shown, while a coil 33 is wound around the magnet 31, the leads of the coil 33 extending back through catheter (not shown). The direction of magnetisation of the magnet 31 can be switched by sending an electrical pulse through the coil, so causing the assembly to take up one of the two magnetisation configurations as in the FIG. 3 example.

1. A catheter guide assembly comprising a magnet support for mounting to a catheter; at least two permanent magnets mounted to the support; and a control system for adjusting the relative magnetisation directions of the permanent magnets between a first configuration in which their directions of magnetisation are such that the magnets generate a relatively large resultant magnetic field, and a second configuration in which their directions of magnetisation are such that the magnets generate a relatively small resultant magnetic field.

2. An assembly according to claim 1, wherein the magnetisation directions of the permanent magnets are aligned in the first configuration.

3. An assembly according to claim 1 or claim 2, wherein the magnetisation directions of the permanent magnets are opposed in the second configuration.

4. An assembly according to any of the preceding claims, wherein the volumes of the permanent magnets are chosen such that in the second configuration, the total volume magnetised in one direction is equal to the total volume magnetised in the opposite direction.

5. An assembly according to any of the preceding claims, wherein 3 permanent magnets are provided.

6. An assembly according to claim 5, wherein two of the permanent magnets are fixed to the support, while the third permanent magnet is moveable relative to the support.

7. An assembly according to any of claims 1 to 5, wherein an electrical coil is provided around one or more of the permanent magnets, the passage of an electric current through the coil causing the magnetisation of the magnet to change.

8. An assembly according to any of the preceding claims, wherein the permanent magnets define a sphere.

9. An assembly according to any of the preceding claims, wherein the support partially surrounds the permanent magnets so as to hold them in place, but permits relative movement between them.

10. An assembly according to any of the preceding claims, wherein the permanent magnets contact one another.

11. An assembly according to any of the preceding claims, wherein the control system comprises at least one control line connected to one of the permanent magnets so that orientation of the permanent magnet can be changed by moving the control line.

12. An assembly according to claim 11, wherein the control system comprises a pair of control lines connected to respective, different parts of the permanent magnet for moving the permanent magnetic in respective opposite directions.

13. A catheter guide assembly substantially hereinbefore described with reference to any of the examples shown in the accompanying drawings.

14. A catheter assembly comprising a catheter; and a catheter guide assembly according to any of the preceding claims mounted to the catheter.

15. A method of performing catheterisation comprising inserting a catheter assembly including a catheter and a catheter guide assembly according to any of claims 1 to 13 mounted to the catheter, with the magnetisation of the permanent magnets set to a first configuration; applying a magnetic field or magnetic field gradient in a controlled manner to cause the catheter to be guided by the catheter guide assembly; setting the magnetisation of the permanent magnets of the catheter guide assembly to their second configuration; removing the controlling magnetic field or magnetic field gradient; and performing a magnetic resonance imaging (MRI) process.
